(19) United States
(12) Patent Application Publication
(10) Pub. No.: US 2006/0223032 A1
(43) Pub. Date: Oct. 5, 2006

(54) Near-infrared transillumination for the imaging of early dental decay

(76) Inventors: Daniel Fried, San Francisco, CA (US); Robert Jones, San Francisco, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/347,637

(22) Filed: Feb. 3, 2006

Related U.S. Application Data
(63) Continuation of application No. PCT/US04/25872, filed on Aug. 6, 2004.
(60) Provisional application No. 60/493,569, filed on Aug. 8, 2003.

(51) Int. Cl. A61C 5/00; A61B 5/117

(52) U.S. Cl. 433/215; 433/114; 433/29; 600/590

(57) ABSTRACT

A method for detecting tooth decay and other tooth anomalies wherein a tooth is transilluminated with a near-infrared light source preferably in the range from approximately 795-nm to approximately 1600-nm, more preferably in the range from approximately 830-nm to approximately 1550-nm, more preferably in the range from approximately 1285-nm to approximately 1335-nm, and more preferably at a wavelength of approximately 1310-nm, and the light passing through the tooth is imaged for determining an area of decay in the tooth. The light source is a fiber-optic bundle coupled to a halogen lamp or more preferably a superluminescent diode, and the imaging device is preferably a CCD camera or a focal plane array (FPA).

### CROSS-REFERENCE TO RELATED APPLICATIONS

This application claims priority from, and is a 35 U.S.C. § 111(a) continuation of, co-pending PCT international application serial number PCT/US2004/025872, filed on Aug. 6, 2004, which designates the U.S., incorporated herein by reference in its entirety, which claims priority from U.S. provisional application Ser. No. 60/493,569, filed on Aug. 8, 2003, the entirety of which is herein incorporated by reference.

This application is related to PCT International Publication Numbers WO 2005/013843 A2 and WO 2005/013843 A3, each of which is incorporated herein by reference in its entirety.

### INCORPORATION-BY-REFERENCE OF MATERIAL SUBMITTED ON A COMPACT DISC

Not Applicable

### STATEMENT REGARDING FEDERALLY SPONSORED RESEARCH OR DEVELOPMENT

This invention was made with Government support under Grant No. 1-R01 DE14698 and Grant No. T32 DE07306-07 awarded by NIH/NICDR. The Government has certain rights in this invention.

### BACKGROUND OF THE INVENTION

1. Field of the Invention

This invention pertains generally to detection of dental caries by transillumination of a tooth, and more particularly to transillumination at wavelengths that are not subject to scattering by sound tooth enamel and identification of dental caries in interproximal sites between teeth.

2. Incorporation by Reference of Publications

The following publications are incorporated by reference herein in their entirety:

J. D. B. Featherstone and D. Young, “The need for new caries detection methods,” Lasers in Dentistry V, San Jose, Calif., Proc. SPIE 3593, 134-140 (1999).

J. Peltola and J. Wolf, “Fiber optics transillumination in caries diagnosis,” Proc Finn Dent Soc, 77, 240-244 (1981).

J. Barenie, G. Leske, and L. W. Ripa, “The use of fiber optic transillumination for the detection of proximal caries,” Oral Surg, 36, 891-897 (1973).

R. D. Holt and M. R. Azeevedo, “Fiber optic transillumination and radiographs in diagnosis of approximal caries in primary teeth,” Community Dent Health, 6, 239-247 (1989).

C. M. Mitropoulis, “The use of fiber optic transillumination in the diagnosis of posterior approximal caries in clinical trials,” Caries Res, 19, 379-384, (1985).

A. Peers, F. J. Hill, C. M. Mitropoulos, and P. J. Holloway, “Validity and reproducibility of clinical examination, fibre-optic transillumination, and bite-wing radiology for the diagnosis of small approximal carious lesions.” Caries Res., 27, 307-311 (1993).

C. M. Pine, “Fiber-Optic Transillumination (FOTI) in Caries Diagnosis,” in Early Detection of Dental Caries, G. S. Stookey, ed., (Indiana Press, Indianapolis, Ind. 1996).

J. Vaarkamp, J. J. t. Bosch, E. H. Verdonschot, and E. M. Bronkhorst, “The real performance of bitewing radiography and fiber-optic transillumination for approximal caries diagnosis,” J Dent Res, 79, 1747-1751 (2000).

A. Schneiderman, M. Elbaum, T. Schultz, S. Keem, M. Greenebaum, and J. Driller, “Assessment of Dental caries with Digital Imaging Fiber-Optic Transillumination (DIFOTI): In vitro Study,” Caries Res., 31, 103-110 (1997).

D. Fried, J. D. B. Featherstone, R. E. Glena, and W. Seka, “The nature of light scattering in dental enamel and dentin at visible and near-IR wavelengths,” Appl. Optics, 34, 1278-1285 (1995).

R. Jones and D. Fried, “Attenuation of 1310 and 1550-nm laser light through dental enamel,” in Lasers in Dentistry VIII, San Jose, Proc. SPIE 4610, 187-190 (June 2002).

G. M. Hale and M. R. Querry, “Optical constants of water in the 200-nm to 200-μm wavelength region,” Appl. Optics, 12, 555-563 (1973).

D. Spitzer and J. J. ten Bosch, “The absorption and scattering of light in bovine and human dental enamel,” Calcif. Tiss. Res., 17, 129-137 (1975).

S. Keem and M. Elbaum, “Wavelet representations for monitoring changes in teeth imaged with digital imaging fiber-optic transillumination,” IEEE Trans Med Imaging, 16, 653-63 (1997).

3. Incorporation by Reference of Patents

The following U.S. patents which describe transillumination techniques and devices are incorporated by reference herein in their entirety:
U.S. Pat. No. 6,341,957
U.S. Pat. No. 6,243,601
U.S. Pat. No. 6,201,880

4. Description of Related Art

During the past century, the nature of dental decay or dental caries has changed dramatically due to the addition of fluoride to the drinking water, the widespread use of fluoride dentifrices and rinses, and improved dental hygiene. Despite these advances, however, dental decay continues to be the leading cause of tooth loss in the United States. By age 17, 80% of children have experienced at least one cavity. In addition, two-thirds of adults in the age range of 35 to 44 have lost at least one permanent tooth to caries. Older adults suffer tooth loss due to the problem of root caries.

Today, almost all new decay occurs in the occlusal pits and fissures of the posterior dentition and the interproximal contact sites between teeth. These early carious lesions are often obscured or “hidden” in the complex and convoluted topography of the pits and fissures or are concealed by debris that frequently accumulates in those regions of the posterior teeth. Such decay, particularly in the early stages, is difficult to detect using the dentist's existing armamentarium of dental x-rays and the dental explorer (a metal mechanical probe). Therefore, new imaging technologies are needed for the early detection of such lesions.

Moreover, the treatment for early dental decay or caries is shifting away from aggressive cavity preparations that attempt to completely remove demineralized tooth structure toward non-surgical or minimally invasive restorative techniques. In non-surgical therapy, a clinician prescribes antibacterial rinses, fluoride treatments, and dietary changes in attempt to naturally remineralize the decay before it becomes irreversible. The success of this type of therapy is contingent on early caries detection and also requires imaging modalities that can safely and accurately monitor the success of such treatment. Conventional x-rays do not precisely measure the lesion depth of early dental decay, and due to ionizing radiation exposure are not indicated for regular monitoring. These constraints and limitations are the impetus for investigating optical imaging systems that could detect early dental decay, while providing the biologically compatible wavelengths that facilitate frequent screening.

Before the advent of x-rays, dentists used light for the detection of caries lesions. In the past 30 years, the development of high intensity fiber-optic illumination sources has resurrected this method for caries detection. Previous groups pursuing visible light transillumination, have used or proposed more advanced imaging techniques like temporal or coherence gating and sophisticated image processing algorithms to enhance the imaging and detection of dental decay.

Fiber-optic transillumination (FOTI) is one technology being developed for the detection of interproximal lesions. One digital-based system, DIFOTITM (Digital Imaging Fiber-Optic Transillumination) from Electro-Optical Sciences, Inc., that utilizes visible light, has recently received FDA approval. During FOTI a carious lesion appears dark upon transillumination because of decreased transmission due to increased scattering and absorption by the lesion. However, the strong light scattering of sound dental enamel at visible wavelengths, 400-nm to 700-nm, inhibits imaging through the tooth.

### BRIEF SUMMARY OF THE INVENTION

The present invention is directed to the detection, diagnosis, and imaging of carious dental tissue. The invention resolves changes in the state of mineralization of dental hard tissues with sufficient depth resolution to be useful for the clinical diagnosis and longitudinal monitoring of lesion progression. One aspect of the invention is to provide system and method for the detection, diagnosis, and imaging of early caries lesions and/or for the monitoring of lesion progression. Another aspect of the invention is to provide a near-infrared transillumination system and method for the detection and imaging of early interproximal caries lesions. A further aspect of the invention is to provide a near-infrared transillumination system and method for the detection of cracks and imaging the areas around composite restorations.

In one mode, near-IR light at 1310-nm is used for the detection and imaging of interproximal caries lesions where a high contrast between sound enamel and simulated lesions is exhibited. In addition, occlusal lesions, root caries, secondary decay around composite restorations, and cracks and defects in the tooth enamel can be seen.

In accordance with one aspect of the invention, a method for detecting tooth anomalies comprises transilluminating a tooth with light having a wavelength in the range from approximately 795-nm to approximately 1600-nm, and the step of imaging light passing through said tooth for determining an anomaly or area of decay in said tooth. In accordance with other aspects of the present invention, a tooth is transilluminated with near-infrared light at a wavelength more preferably in the range from approximately 830-nm to approximately 1550-nm, more preferably in the range from approximately 1285-nm to approximately 1335-nm, and more preferably at a wavelength of approximately 1310-nm.

In another mode, the light is filtered to remove extraneous light. The light may be polarized with one or more polarizing filters to remove light not passing through said tooth. The polarizing filters are preferably crossed high-extinction polarizing filters. The method may also comprise filtering said light with a bandpass filter to remove light outside a specified bandwidth.

Generally, transilluminating a tooth comprises directing light from a near-infrared light source at a surface of said tooth. The light source may be a fiber-optic bundle coupled to a halogen lamp, a superluminescent laser diode, or similar IR source.

In one mode of the invention, the light source may be manipulated behind the tooth to direct said light at a lingual surface of the tooth. Alternatively, the light source may be manipulated in front of said tooth to direct said light at a facial surface of the tooth.

In one embodiment, the step of imaging light passing through the tooth comprises detecting intensity of light passing through the tooth at a plurality of spatial positions, developing a spatial profile of the detected light intensity, using the spatial intensity profile to identify an area in said tooth exhibiting intensity gradients, designating said area of said tooth exhibiting intensity gradients as an area of tooth decay. In another embodiment, detected light intensity is compared over at least a portion of said spatial positions for determining an area of decay in said tooth and an area of the tooth exhibiting a lower detected light intensity than an at least partially surrounding area is designated as an area of tooth decay.

In one aspect of the invention, the step of detecting the intensity of light passing through said tooth comprises directing a first detector at an aspect of the tooth, such as a facial aspect of the tooth, an occlusal aspect of the tooth, an opposite aspect of the tooth from the light source, or the same aspect of the tooth as the light source.

According to another embodiment of the invention, a second detector a second detector may at a different aspect of the tooth than the first detector. For example, the second detector may be directed at an occlusal aspect of the tooth while the first detector is directed at a facial aspect of the tooth. The detector may comprise a focal plane array, near-infrared CCD camera, or the like.

The method may be used to determine anomalies such as an area of decay, a crack, a composite restoration, and dental caries in an occlusal site or interproximal contact site between said tooth and an adjacent tooth of said tooth.

According to another aspect of the invention, a system for detecting tooth decay comprises a near-infrared light source emitting light having a wavelength in the range from approximately 785-nm to approximately 1600-nm wherein the light source is configured to transilluminate a tooth, and means for imaging light passing through said tooth and determining an area of decay in said tooth. In accordance with other aspects of the present invention, a light source has a wavelength more preferably in the range from approximately 830-nm to approximately 1550-nm, more preferably in the range from approximately 1285-nm to approximately 1335-nm, and more preferably at a wavelength of approximately 1310-nm. In one mode, the light source comprises a polarized light source. In another mode, the light source comprises an unpolarized light source. In one embodiment, the light source comprises a fiber-optic bundle coupled to a halogen lamp. In another embodiment, the light source comprises a superluminescent diode (SLD). In still another embodiment, the imaging means comprises a CCD camera. In another embodiment, the imaging means comprises a focal plane array (FPA).

According to yet another aspect of the invention, a system for detecting a tooth anomaly comprises a near-infrared light source having a wavelength in the range from approximately 795-nm to approximately 1600-nm, wherein the light source is configured to transilluminate a tooth. The system further includes an imaging device configured to detect intensity of light from said light source passing through said tooth, whereby an anomaly in said tooth can be determined from intensity of light detected by said imaging device.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is graph comparing the attenuation coefficient of dental enamel and water as a function of wavelength.

FIG. 2 is a flowchart of an embodiment of a method for detecting dental caries by near-infrared transillumination according to the present invention.

FIG. 3 is a schematic diagram of a system for Near-Infrared Transillumination of whole teeth and tooth sections according to the present invention.

FIG. 4 is a schematic diagram of another system for Near-Infrared Transillumination of whole teeth and tooth sections according to the present invention using two light sources.

FIGS. 5A-5D are views of a tooth with a simulated lesion. FIG. 5A is a side view of a 3-mm thick tooth section with a simulated lesion. FIG. 5B illustrates that the lesion cannot be seen using transillumination with visible light and a CCD camera. FIG. 5C illustrates that the lesion is clearly visible under NIR. FIG. 5D is an x-ray of the section using D-speed film indicates the small contrast difference between the simulated lesion and sound enamel.

FIGS. 6A-6F are NIR transillumination images of tooth sections with simulated lesions are shown for sample thicknesses of 2-mm, 3-mm, 4-mm, 5-mm, 6-mm and 6.75-mm, respectively. The corresponding spatial line profiles are shown on the inset in the lower right of each image, and the measured lesion contrast is shown in the lower left. The left axis represents the pixel intensity ranging from 0 to 4096, and the bottom axis the pixel position through the lesion.

FIG. 7 is a graph showing the mean ±s.d lesion contrast plotted versus thickness of plano-parallel enamel samples, n=5.

FIG. 8 is an NIR image of a whole tooth sample. A natural carious lesion and a composite restoration are seen on the left and right, respectively. The tooth is slightly rotated to present different viewing angles. A crack is also visible in the center of the tooth.

### DETAILED DESCRIPTION OF THE INVENTION

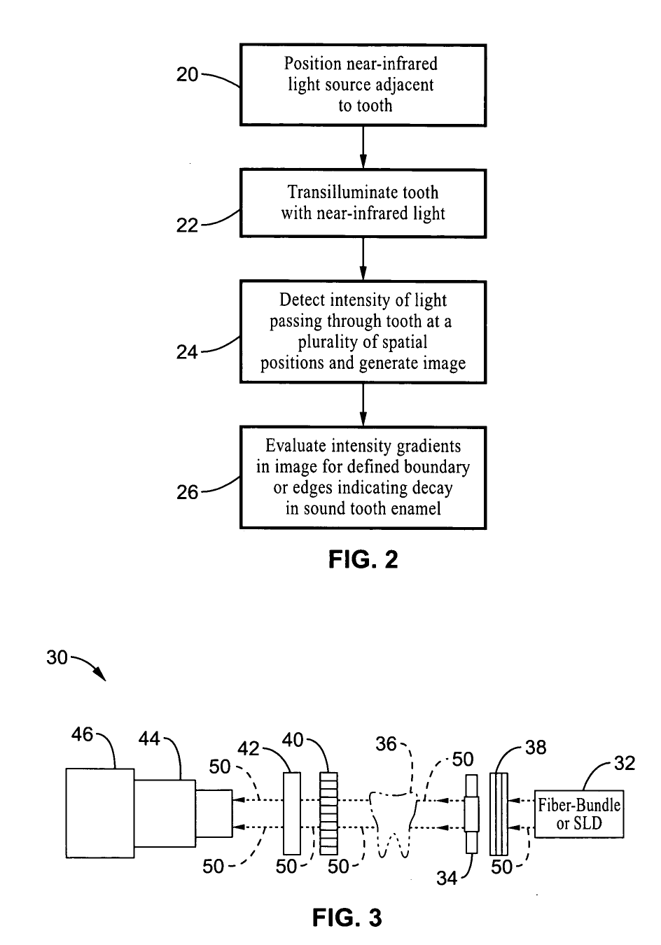

Referring more specifically to the drawings, for illustrative purposes the present invention is embodied in the system(s) and method(s) generally shown in FIG. 2 through FIG. 8. It will be appreciated that the apparatus may vary as to configuration and as to details of the parts, and that the method may vary as to the specific steps and sequence, without departing from the basic concepts as disclosed herein.

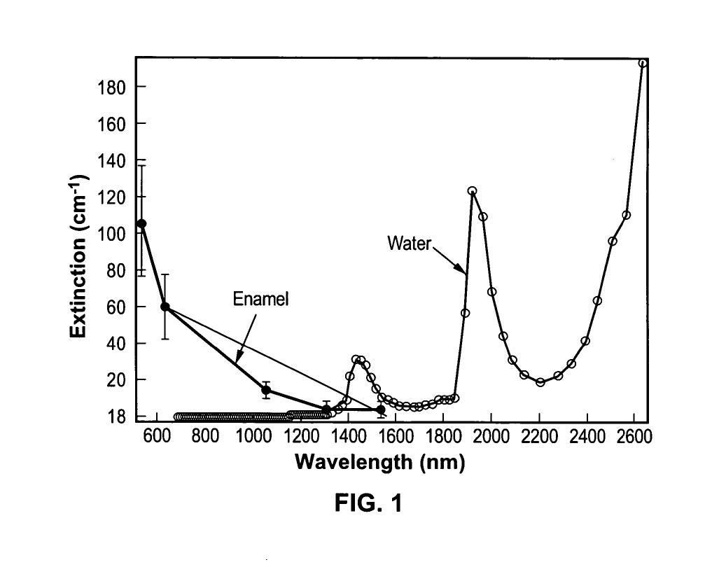

A principal limiting factor of light in the visible wavelength range from approximately 400-nm to 700-nm being transmitted through a tooth is light scattering in sound enamel and dentin. The present invention overcomes that limiting factor by employing near-infrared (NIR) for transillumination of a tooth. The magnitude of light scattering in dental enamel decreases as 1/λ³, where λ is the wavelength, due to the size of the principal scatterers in the enamel. The attenuation coefficients of dental enamel measured at 1310-nm and 1550-nm were 3.1 cm⁻¹ and 3.8 cm⁻¹, respectively. As shown in FIG. 1, the magnitude of scattering at those wavelengths is more than a factor of 30 times lower than in the visible range. This translates to a mean free path of 3.2 mm for 1310-nm photons, indicating that enamel is transparent in the near-infrared (NIR). At longer wavelengths past 1550-nm, the attenuation coefficient is not expected to decrease any further due to the increasing absorption coefficient of water, 12% by volume, in dental enamel.

As indicated above, at shorter wavelengths the light is subject to scattering. On the other hand, at longer wavelengths, absorption of water in the tissue increases and thereby reduces the penetration of infrared light.

Note also that, during the caries process, micropores are formed in the lesion due to partial dissolution of the individual mineral crystals. Such small pores can behave as scattering centers smaller than the wavelength of the light. Accordingly, there can be an increase in both the magnitude of light scattering and the contribution of large angle scattering to the scattering phase function in caries lesions due to the increased microporosity. Changes in the optical constants and scattering phase function of enamel and dentin result in more rapid depolarization of incident polarized light. Accordingly, polarized light (e.g., via linear or circular polarization) will provide a greater image contract than unpolarized light and can be exploited to aid in the near-infrared optical detection of carious lesions.

The present invention is particularly useful in detecting occlusal caries (biting surfaces) and interproximal caries or lesions located at interproximal contact sites between adjacent teeth. The present invention is also useful in detecting other anomalies such as root caries, cracks, and imaging around composite restorations.

Referring to FIG. 2, an exemplary method for detecting tooth anomalies such as dental decay or caries according to the invention is illustrated. First, a near-infrared light source is positioned adjacent to a tooth to be examined, as shown at block 20. Next, the tooth is transilluminated with the near-infrared light, as shown at block 22. The wavelength of the light is preferably in the range from approximately 795-nm to approximately 1600-nm, more preferably in the range from approximately 830-nm to approximately 1550-nm, more preferably in the range from approximately 1285-nm to approximately 1335-nm, and more preferably at a wavelength of approximately 1310-nm. Use of near-infrared light in these ranges provides deeper depth resolution and improved contrast between sound and carious enamel as compared to light at other wavelengths.

Once the tooth is transilluminated, the intensity of the light passing through the tooth at a plurality of spatial positions is detected, thereby forming an image of the tooth structure, as shown at block 24. The detected light intensity over at least a portion of the spatial positions is then compared so that an area of tooth decay can be identified, as shown at block 26. This is preferably accomplished by developing a spatial profile so that intensity gradients can be seen. An area of the tooth that exhibits a lower detected light intensity than an at least partially surrounding area is indicative of an area of tooth decay. While contrast alone can be used as an indicator of tooth decay, more preferably the existence of a defined boundary or edge between areas exhibiting intensity gradients is a more accurate indicator. It will be appreciated, of course, that a dentist or trained clinician will review and evaluate the images to distinguish lesions from, for example, areas containing fillings, composite restorations, or other non-dental caries areas that effect intensity gradients in the image. Note that the incident light is preferably linearly polarized and, preferably, only light in the orthogonal polarization state is measured.

An exemplary NIR imaging device 30 is shown schematically in FIG. 3. Light 50 is emitted from a light source 32, through polarizer 38 and aperture 34 toward tooth or series of teeth 36. Light source 32 preferably comprises a broadband light source, such as fiber-optic bundle coupled to a halogen lamp, or a superluminescent laser diode (SLD). It was found that the speckle of conventional narrow bandwidth diode lasers such as a 50-mW 1310-nm source, Model QLD-1300-50 (Qphotonics Inc., Chesapeake, Va.) interfered significantly with image resolution and were not optimal for the present invention.

Crossed near-IR polarizers, 38, 40 are used to remove light that directly illuminated the array without passing through the tooth. In a clinical situation, the light passing between the teeth will saturate the image preventing detection. Dental enamel is birefringent and, therefore, the polarization state of the light passing through the tooth may be altered to reduce extinction. Polarization gating using crossed high extinction polarizers 38, 40 removes extraneous light that does not pass through the tooth and exploits the native birefringence of the tooth enamel to rotate the plane of polarization so that only light that passes through the tooth is measured. Caries lesions depolarize light which provides better image contrast between sound and carious tissue

Light passing through tooth 36 and polarizer 40 is further filtered with bandpass filter 42 to remove all light outside the spectral region of interest.

The light is then focused with lens 44 and picked up with detector 46 to acquire images of tooth or teeth 36. In a preferred embodiment, detector 46 comprises a near-infrared (NIR) InGaAs focal plane array (FPA).

The illuminating light intensity of light source 32, the diameter of aperture 34, and the distance of the light source to tooth 36, may all be adjusted to obtain the maximum contrast between the lesion and the surrounding enamel without saturation of the InGaAs FPA around the lesion area.

Alternatively, detector 46 may comprise a CCD camera with the IR filter 42 and a 70-nm bandpass filter centered at approximately 830-nm. Alternatively, the bandpass filter may be removed. Imaging with a near-IR CCD camera is less expensive with an InGaAs detector, but does not perform as well as an InGaAs detector. As another alternative, transillumination can also be conducted using a CCD camera with a near-infrared phosphor in the range of approximately 1000-nm to approximately 1600-nm.

In yet another alternative embodiment, image quality may be improved by utilizing biocompatible index matching fluids and gels and/or solid materials of high refractive index to reduce reflection, total internal reflection, and refraction at the tooth entrance and exit surfaces. Such materials would be placed on the end of the illumination source 32 and/or the detector 46 and would make physical contact with the tooth surface

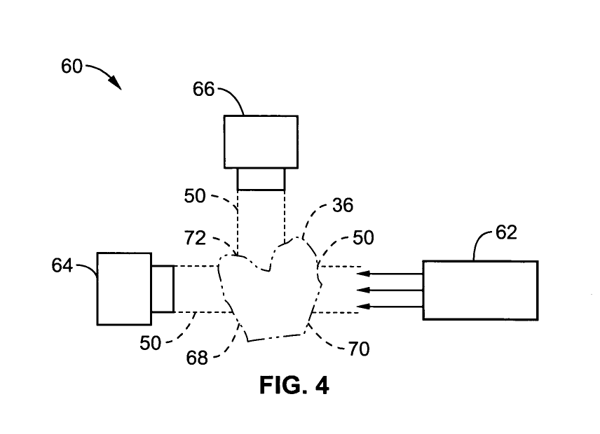

Now referring to FIG. 4, an alternative embodiment of NIR imaging device 60 is shown schematically for imaging tooth 36. This device 60 may be used for the near-IR imaging of occlusal and pit and fissure lesions by placing light source 62 on the facial aspect 68 or lingual aspect 70 of the tooth and placing a second imaging source 66 above the occlusal surface 72 of the tooth 36 in addition to the first imaging source 68 either the facial or lingual aspects, 68, 70. Detection of light 50 along different axes may be achieved with a combination of prisms, mirrors or optical fiber components. For example, the imaging fiber optic bundle 62 could be fitted with a 900 prism (not shown) and connected to a near-IR imaging camera. Alternatively, the light source may also be placed in any combination of these viewing angles, including having the light source and imager on the same aspect of the tooth.

### EXAMPLE 1

### Sample Preparation

Thirty plano-parallel sections of enamel of various thicknesses (2-mm, 3-mm, 4-mm, 5-mm, 6-mm, and 6.75-mm) were prepared from non-carious human teeth. These sections were stored in a moist environment to preserve tissue hydration with 0.1% thymol added to prevent bacterial growth. Uniform scattering phantoms simulating dental decay were produced midway through each section by drilling 1-mm diameter×1.2-mm deep cavities in the proximal region of each sample and filling the cavities with hydroxyapatite paste. A thin layer of unfilled composite resin was applied to the outside of the filled cavity to seal the hydroxyapatite within the prepared tooth cavity.

### NIR Imaging

Both a 150-watt halogen lamp, Visar™ (Den-Mat, Santa Maria, Calif.), and a 1310-nm superluminescent diode (SLD) with an output power of 3.5 mW and a bandwidth of 25-30 nm, Model QSDM-1300-5 (Qphotonics Inc., Chesapeake, Va.) were separately used as the illumination source.

Model K46-252 (Edmund Scientific, Barrington, N.J.) crossed near-IR polarizers were used to remove light that directly illuminated the array without passing through the tooth. A 50-nm bandpass filter centered at 1310-nm Model BP-1300-090B (Spectrogon US, Parsippany, N.J.) was used to remove all light outside the spectral region of interest.

A near-infrared (NIR) InGaAs focal plane array (FPA) having a resolution of 318×252 pixels was used to acquire all of the images. The particular FPA used was an Alpha NIRTM (Indigo Systems, Goleta, Calif.) with an Infinimite™ lens (Infinity, Boulder, Colo.).

The acquired 12-bit digital images were analyzed using IRVista™ software (Indigo Systems, Goleta, Calif.).

The illuminating light intensity, source to sample distance, and the aperture diameter were adjusted for each sample to obtain the maximum contrast between the lesion and the surrounding enamel.

Although the 3.5-mW SLD source provided similar image quality to the halogen lamp source, all the images illustrated herein were acquired using the fiber-optic illuminator. Due to the natural tooth contours, the sides near the simulated lesions in the tooth sections were masked with putty to ensure that light traveled the full width of the sample. This masking is not applicable in a clinical situation and was not necessary to acquire images of whole teeth.

In addition, good images of teeth were obtained using the 3.5 mW SLD operating at 1310-nm. This is important because this illumination source is very compact and can be easily placed in the oral cavity. Furthermore, the SLD is much more compact than the illumination source used for DiFOTI and can be integrated into a small dental explorer and manipulated behind the teeth for collection of images using the camera.

### Visible and X-ray Imaging

A tooth section of minimal sample thickness, 3-mm, was chosen for comparison of the NIR transillumination system with conventional visible light FOTI and x-ray transillumination. For visible light transillumination, the same fiber-optic illuminator was used to illuminate the section and a color ⅓″ CCD camera with a resolution of 450 lines, Model DFK 5002/N, (Imaging Source, Charlotte, N.C.) equipped with the same Infinimite™ lens recorded the projection image. The corresponding x-ray image was acquired by placing the section directly on Ultra-Speed™ D-speed film (Kodak, Rochester, N.Y.) using 75 kVp, 15 mA, and 12 impulses.

### Image Analysis

The coordinates of each simulated lesion were known prior to analyzing the contrast of each lesion. The mean pixel intensity of the lesion and the enamel above and below the lesion was measured using the IRvista™ software. Lesion contrast was calculated for each sample as follows:
Lesion Contrast (C)=(I_{E}−I_{L})/I_{E},
where I_{E} is the mean intensity of the enamel bordering the lesion and I_{L} is the mean intensity of the lesion. Lesion contrast is defined as a ratio that will vary from zero (0) to one (1). For each of the six sample thicknesses measured, the mean lesion contrast was calculated and plotted versus sample thickness.

Although contrast is important, the boundary or edge between the lesion and the sound tooth structure is central to detection of the lesion. Therefore, the spatial intensity profile of a lesion with its surrounding enamel was analyzed. An intensity profile was mapped from a (1) distinct line in six sample images representing each thickness.

### Results

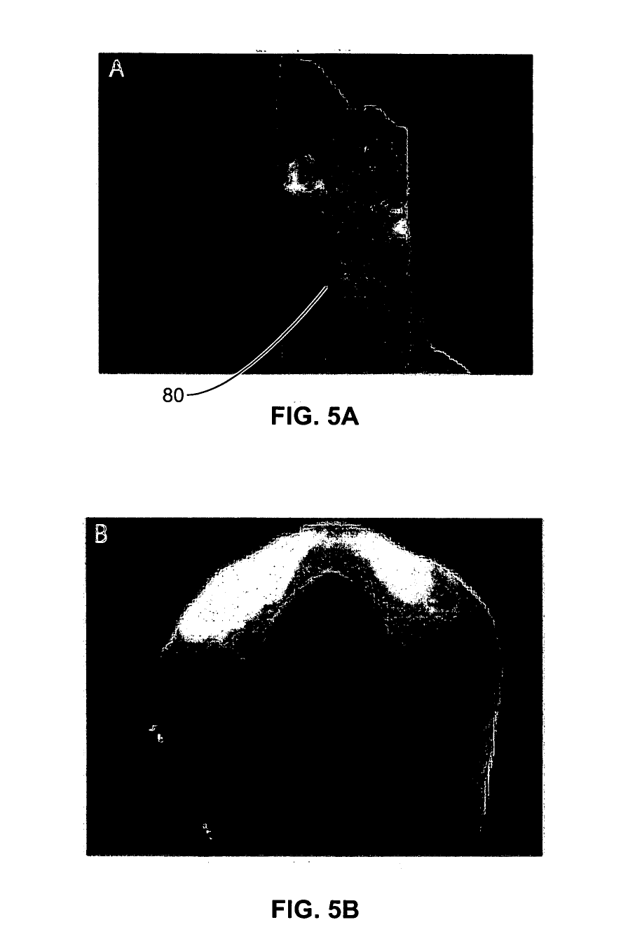

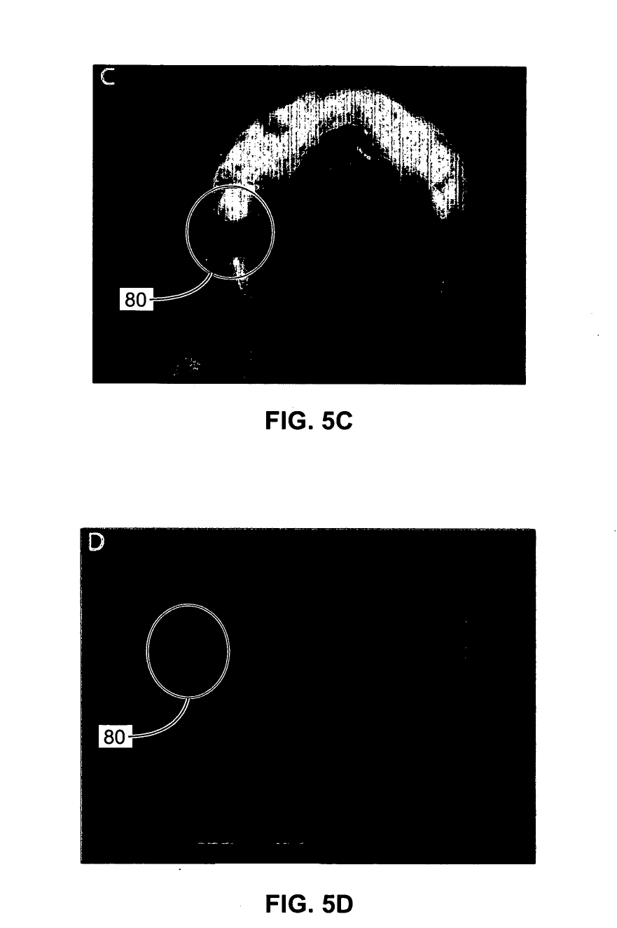

Visible light, NIR and X-ray images of a simulated lesion placed in one of the 3-mm thick tooth sections are shown in FIGS. 5A-5D. The lesion 80 cannot be seen using visible light transillumination, however the lesion is clearly visible with high contrast using NIR light transillumination. A radiographic image of the tooth section using D-speed film shows a low lesion contrast, or a small contrast difference between the lesion and the surrounding enamel.

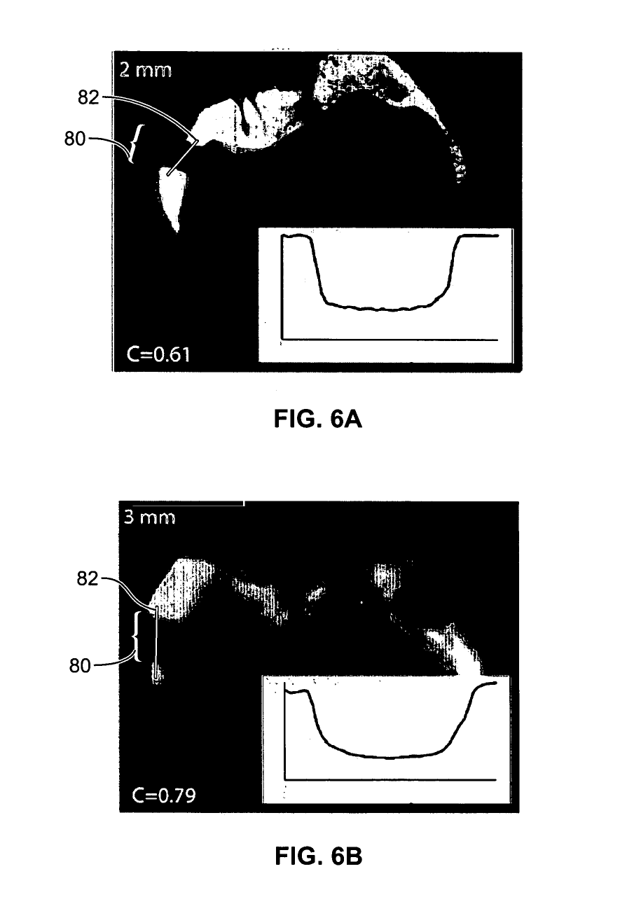

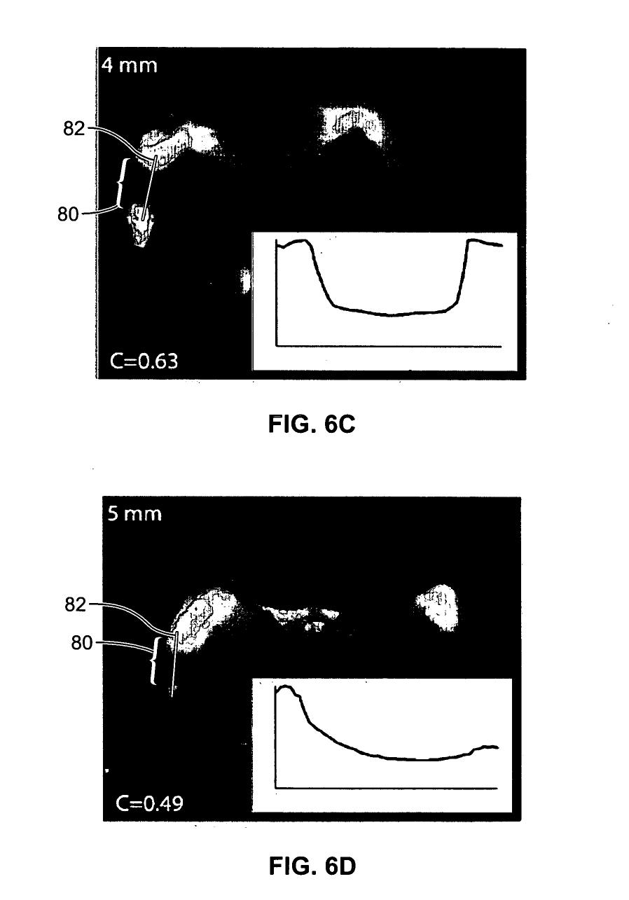

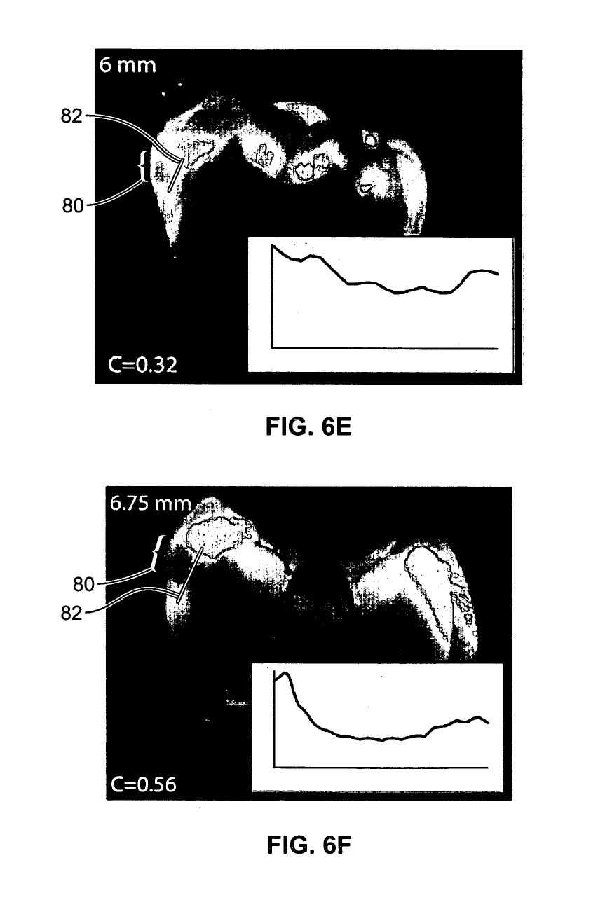

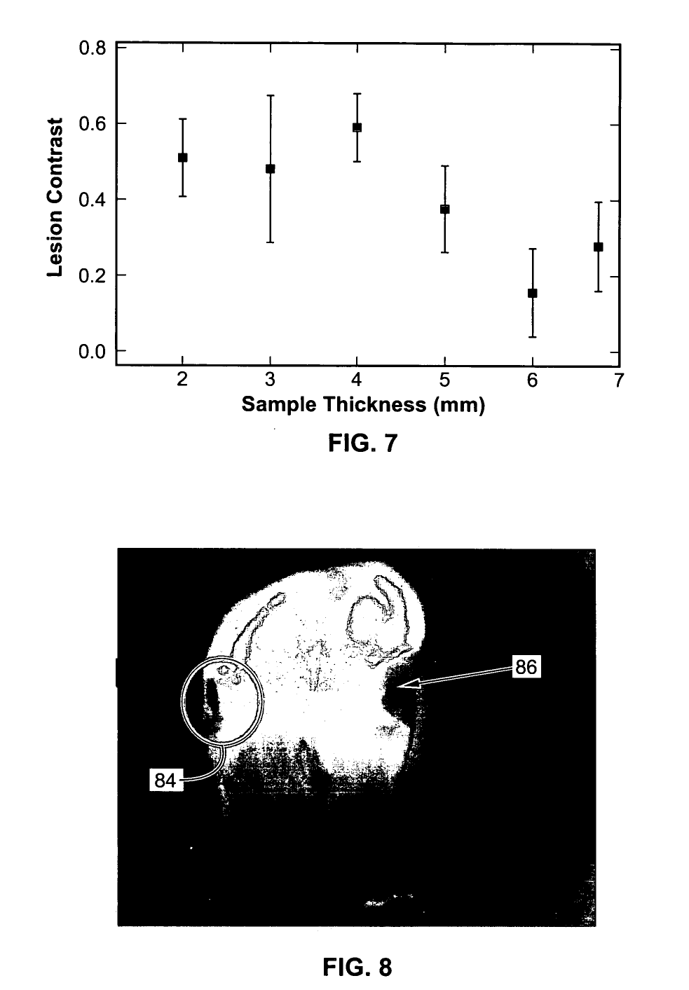

The lesion contrast was calculated for all thirty of the enamel sections under NIR illumination. Representative spatial intensity profiles from six of the samples of each thickness and the corresponding images are shown in FIGS. 6A-6F. From these profiles, the edge or boundary between the sound enamel and the lesion is clearly demarcated in all six of the sections. The image contrast plotted vs. section thickness is shown in FIG. 7. A lesion contrast of greater than 0.35 was seen in all the sections with the exception of the 6-mm samples. A 0.35 lesion contrast is equivalent to a lesion intensity that is 65% of the surrounding enamel.

For 6-mm samples, a mean lesion contrast of 0.16 was calculated. A steep intensity gradient is visible between the surrounding enamel and the lesion. This gradient is less pronounced for sections greater than 4-mm thick, especially on the lower border of the lesion. A NIR image of a whole tooth sample with a natural lesion 84, depicted in FIG. 8, illustrates that a natural lesion 84 can be resolved with the same success as the simulated lesions placed in plano-parallel sections. A composite filling 86 is also visible on the opposite side of the tooth in FIG. 8, indicating that there is also high contrast between composite filling materials and sound tooth structure.

### Discussion of Experimental Results

The high contrast and intensity profiles of the simulated lesions with the surrounding enamel indicate the significant potential of NIR transillumination for imaging dental caries. Since the clinical use of transillumination is to detect interproximal lesions, it is important to note that forty of the sixty-four interproximal surfaces in the mouth would require imaging through less than 5-mm of enamel. This study suggests that resolving caries lesions through 5-mm of enamel is clinically feasible. This is further demonstrated by the NIR imaging of whole teeth with natural decay.

During the transillumination of whole tooth samples, polarization gating with crossed polarizers was critical for preventing the illuminating light from saturating the InGaAs array near the area of the lesion “shadow”. This technique will also be important in a clinical setting where adjacent tooth surfaces will reflect, but not depolarize the light, and could interfere with the accuracy of the projection image.

During demineralization of enamel in the caries process, preferential dissolution of the mineral phase creates pores that highly scatter light. The simulated lesions in our study are primarily made up of isotropic scatterers, with scattering occurring at the grain boundaries in the hydroxyapatite powder. Therefore, such simulated lesions may possibly overestimate the magnitude of scattering in natural caries lesions; however, creating more accurate optical simulated lesions requires an intimate understanding of the fundamental optical properties of carious tissue that has yet to be determined.

It was found that 1310-nm is optimal for both high transmission through sound dental enamel and for achieving high contrast between caries lesions and sound enamel.

Simulated lesions composed of an unorganized paste of hydroxyapatite, strongly scatter the 1310-nm light, which provides high contrast with the transparent sound enamel. Optical transillumination is similar to other projection imaging modalities like conventional x-rays, however the image contrast arises from changes in tissue scattering as opposed to variations in tissue density. Therefore, this method can be more sensitive than x-rays for resolving early caries lesions. Clinicians are trained to diagnose at the low lesion contrast depicted in the radiograph of FIG. 5D, but the high contrast in the NIR image suggests that the simulated lesions are more sensitive to optical detection. This is due to the fact that the simulated lesions have only slightly lower density than the sound enamel but strongly scatter NIR light.

In addition, favorable images to a depth of 4-mm to 5-mm were obtained using a CCD camera with the IR filter removed operating at approximately 830-nm.

As can be seen, therefore, there are several advantages between the present invention and known systems that use DiFOTI or other FOTI techniques. These include:

(a) Illumination

The DiFOTI system and other FOTI systems utilize an unfiltered fiber-optic illuminator with most intensity in the visible range, as opposed to the broadband near-IR illumination sources of the present invention. Tests revealed that narrow band sources such as conventional laser diodes generate too much laser speckle for imaging. Successful results were achieved with a fiber-optic illuminator having either a 50-nm bandpass filter centered at 1310-nm or a 70-nm bandpass filter centered at 830-nm. Test results were also favorable (speckle-free) with a low cost 3.5 mW, single mode fiber pigtailed, superluminescent laser diode operating at 1310-nm with a bandwidth of 25-nm to 30-nm.

(b) Image Processing

DiFOTI utilizes proprietary image processing techniques to improve image quality. Although imaging processing techniques may be used in conjunction with the current invention, post imaging digital processing methods is generally not required to improve performance.

(c) Performance

The images collected with FOTI and DiFOTI are not true projection or transillumination images, since the penetration of visible light or the mean-free path is less than 100-μm in enamel. The way these systems work is that light migrates through the enamel of the tooth, backlighting the lesion for better contrast. That means that these systems must have a direct line of sight to the lesion surface. Therefore, they cannot be used to determine how far a lesion has penetrated through the enamel since they can only view the lesion surface.

The present invention acquires true projection images similar to x-rays by imaging through the full thickness of the enamel. In those images, the camera does not have a direct line of site to the lesion surface. This is possible because of the increase in the mean free path of enamel, that is optimum at 1310-nm-3.3 mm.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A method for detecting tooth anomalies, comprising:
transilluminating a tooth with light having a wavelength in the range from approximately 795-nm to approximately 1600-nm; and
imaging light passing through said tooth for determining an anomaly in said tooth.

2. A method as recited in claim 1, wherein said light has a wavelength in the range from approximately 830-nm to approximately 1550-nm.

3. A method as recited in claim 1, wherein said light has a wavelength in the range from approximately 1285-nm to approximately 1335-nm.

4. A method as recited in claim 1, wherein said light has a wavelength of approximately 1310-nm.

5. A method as recited in claim 1, further comprising:
filtering said light to remove extraneous light.

6-8. (canceled)

9. A method as recited in claim 1, wherein transilluminating a tooth comprises directing light from a near-infrared light source at a surface of said tooth.

10-13. (canceled)

14. A method as in claim 1, wherein transilluminating a tooth comprises simultaneously directing light from a near-infrared light source at a surface of a plurality of teeth.

15-25. (canceled)

26. A method as in claim 1, wherein imaging light passing through said tooth comprises determining an area of decay in said tooth.

27. A method as in claim 1, wherein imaging light passing through said tooth comprises determining a crack in said tooth.

28. A method as in claim 1, wherein imaging light passing through said tooth comprises determining an anomaly around a composite restoration in said tooth.

29-30. (canceled)

31. A method of detecting tooth decay, comprising:
transilluminating a tooth with a near-infrared light source having a wavelength in the range from approximately 795-nm to approximately 1600-nm;
detecting intensity of light passing through said tooth at a plurality of spatial positions;
comparing detected light intensity for at least a portion of said spatial positions; and
designating an area of said tooth exhibiting a lower detected light intensity than an at least partially surrounding area as an area of tooth decay.

32. A method of detecting tooth decay, comprising:
transilluminating a tooth with a near-infrared light source having a wavelength in the range from approximately 795-nm to approximately 1600-nm;
detecting intensity of light passing through said tooth at a plurality of spatial positions;
developing a spatial profile of said detected light intensity;
using said spatial intensity profile, identifying areas in said tooth exhibiting intensity gradients; and
designating said area of said tooth exhibiting intensity gradients as an area of tooth decay.

33. (canceled)

34. A method as in claim 31, wherein said light has a wavelength in the range from approximately 830-nm to approximately 1550-nm.

35. A method as in claim 31, wherein said light has a wavelength in the range from approximately 1285-nm to approximately 1335-nm.

36. A method as in claim 31, wherein said light has a wavelength of approximately 1310-nm.

37. A method as in claim 31, further comprising:
filtering said light with one or more polarizing filters to remove extraneous light not passing through said tooth.

38. A method as in claim 31, wherein transilluminating a tooth comprises directing light from a near-infrared light source at a surface of said tooth.

39-67. (canceled)

68. A method as in claim 1, wherein said light has a wavelength in the range from approximately 830-nm to approximately 1550-nm.

69. A method as in claim 1, wherein said light has a wavelength in the range from approximately 1285-nm to approximately 1335-nm.

70. A method as in claim 1, wherein said light has a wavelength of approximately 1310-nm.
